(19) United States
(12) Patent Application Publication
(10) Pub. No.: US 2004/0254433 A1
(43) Pub. Date: Dec. 16, 2004

(54) Sensor introducer system, apparatus and method

(76) Inventors: Steven D. Bandis, West Jordan, UT (US); Robert W. Hitchcock, Sandy, UT (US)

Correspondence Address: TRASK BRITT, P.O. BOX 2550, SALT LAKE CITY, UT, 84110, US

(21) Appl. No.: 10/461,793

(22) Filed: Jun. 12, 2003

(51) Int. Cl. A61B 5/05

(52) U.S. Cl. 600/347

(57) ABSTRACT

An assembly including an introducer cannula and subcutaneous measurement device, and a method of its use. The device can form an aseptic seal to the introducer to resist passage of pathogens through the cannula. Insertion of the device into the introducer desirably causes a one-way coupling operable to force removal of the introducer and device as a single assembly. Installation of certain devices into certain introducers forms an electrical connection between an electrode carried by the introducer and system electronics. Desirably, the introducer provides a funnel-like structure operable to guide a device's probe tip into reception in the introducer's cannula.

### TECHNICAL FIELD

This invention relates generally to medical devices and methods for measuring a variable to indicate a state, or condition, in a human body. Certain apparatus according to the instant invention can be embodied facilely to install and anchor an implantable biosensor in an aseptic manner. One application includes measuring glucose for ongoing diabetes management.

### BACKGROUND

Treatment and management of diabetes is undertaken through many and varied techniques. The approach predominantly used for self-monitoring of blood glucose involves periodic pricks of the skin with a needle, whereby a blood sample is obtained and tested directly to provide information about blood glucose levels. This information is then utilized as a basis from which to approximate the administration of insulin to maintain glucose equilibrium within the patient. While such a direct measurement of glucose levels in periodic blood samples from diabetes patients provides reasonably useful information about insulin levels at a given point in time, the dynamic nature of blood glucose chemistry and the complexity of factors influencing blood sugar levels renders such information less than optimal.

Glucose level in the subcutaneous interstitial fluid very closely approximates the glucose level in the blood, with a negligible lag. The variables of patient food selection, physical activity and insulin dosage, regime and protocol for a person with diabetes each have a dynamic impact on physiologic balance within the patient's body that can change dramatically over a short period of time. If the net result of changes in these variables and dynamics results in disequilibrium expressed as too much glucose (“hyperglycemia”), more insulin is required, whereas too little glucose (“hypoglycemia”), requires immediate intervention to raise the glucose levels. A deleterious impact on physiology follows either such disequilibrium.

Hyperglycemia is the source of most of the long-term consequences of diabetes, such as blindness, nerve degeneration, and kidney failure. Hypoglycemia, on the other hand poses the more serious short-term danger. Hypoglycemia can occur at any time of the day or night and can cause the patient to lose consciousness, necessitating frequent monitoring of blood glucose levels that renders the skin-prick approach tedious, painful and in some cases impractical. Even diligent patients who perform finger-sticking procedures many times each day achieve only a poor approximation of continuous monitoring. Accordingly, extensive attention has been given to development of improved means of monitoring patient glucose levels for treatment of diabetes. More recently, many efforts to continuously monitor glucose levels have involved implantable electrochemical biosensors, necessitating improved means for introducing such biosensors.

Heretofore, introduction of such biosensors has in some instances been accomplished in conjunction with catheters and/or needles. Some configurations include incorporating sensors within catheter and needle structures. Other configurations include passing sensors through catheters or needles. Configurations that provide for the passage of sensors through catheters or needles have been adapted from fluid infusion devices and as such have not addressed the specific needs of a sensor introducer system.

Typical introduction means for both fluid and sensor introduction of the prior art are disclosed in various publications including certain United States patent documents. The following relevant documents exemplify the prior art: “Subcutaneous Infusion Device” to Mejslov, U.S. Pat. No. 6,123,690; “Subcutaneous Implantable Sensor Set Having the Capability to Remove Deliver Fluids To An Insertion Site” to Mastrototaro et al., U.S. Pat. No. 5,951,521; “Transdermal Introducer Assembly” to Mastrototaro et al., U.S. Pat. No. 5,779,665; “Transcutaneous Sensor Insertion Set” to Cheney, II at al., U.S. Pat. No. 5,568,806; and “Infusion Set For An Intermittent or Continuous Administration of a Therapeutical Substance” to Teissen-Simony, U.S. Pat. No. 5,522,803. An application for a US patent, Ser. No. 10/401,224, titled “IMPLANTABLE BIOSENSOR SYSTEM, APPARATUS AND METHOD”, was filed on Mar. 26, 2003, and is incorporated herein as though set forth in its entirety for its teachings of certain biosensors operable in the instant invention.

One problem associated with sensor introducers is the difficulty of facilely establishing them at a selected site. Previously, needles have been associated with sensors to provide rigidity to a relatively less rigid sensor-carrying cannula to enable delivery. While a rigid needle is well suited to facilitate delivery of sensor probes, such a configuration alone is essentially contextually incomplete; more complete previous systems have anticipated the need to include at least means of anchoring the system to a patient.

Prior art sensor introducer systems with anchoring hubs have lacked adequate means of guiding probes such as sensors into position. Those few introducer systems intended for the through passage of sensors have not adequately addressed the needs for guiding the sensor structure into position, providing an infection barrier between the sensor and the introducer and non-releasably attaching the sensor assembly to the introducer catheter assembly.

To provide continuous measurement, one or more biosensors can be placed at various locations within the body. One method of placement is percutaneously with an indwelling sensor and an attached external wire associated with a readout device. A risk of infection is associated with percutaneous introducers, and they must typically be replaced at regular and frequent intervals because of the risk of infection at the insertion site. An additional risk of infection can occur through the lumen of the introducer catheter. While prior art fluid infusion devices such as those disclosed in the '690 patent provide a self-sealing septum in an attempt to partially address the risk of infection, it is impractical to use this type of septum seal in conjunction with a flexible probe that cannot pierce such a septum.

A need remains for a sensor wherein a miniaturized probe of suitable materials and characteristics may facilely be placed percutaneously. A need also exists for an introducer incorporating a guiding structure in a housing suitable to readily guide a flexible sensor probe into a catheter lumen in order to facilitate its placement at the patient site. There is also a need to achieve an attachment of the sensor with structure associated with the introducer to prevent either inadvertent or intentional decoupling. Further needed is a way to limit the potential for infection to the patient through the housing and catheter. Such an infection limiting construction desirably would not interfere with attachment of the sensor to the introducer.

### BRIEF SUMMARY OF THE INVENTION

The invention provides an assembly including an introducer cannula and a subcutaneous measurement device, and a method of its use. In certain embodiments, the measurement device can form an aseptic seal to the introducer to resist passage of pathogens through the cannula. Insertion of the device into the introducer can join a one-way coupling operable to force removal of the introducer and device as a single assembly. Installation of certain devices into certain introducers also forms an electrical connection between an electrode, carried by the introducer, and system electronics. In some embodiments, the introducer provides a funnel-like structure operable to guide a measurement device's probe tip into reception in the introducer's cannula.

An introducer is used to provide subcutaneous access into a subject, or medical patient, for a measurement device. An introducer typically includes a cannula and a housing. The cannula has a lumen extending between a proximal end and a distal end, with the proximal end being associated with affixing structure adapted to resist motion of the proximal end relative to a skin surface of the subject. The housing can be arranged to dispose a guide structure in association with the proximal end of the lumen. A housing can also include structure operable to couple with a measurement device.

Guide structure generally facilitates insertion of a biosensor or other measurement device's probe into the introducer cannula. The currently preferred guide structure provides a conduit having a first opening, disposed at a first end, that is sized larger than a size of an opening at the proximal end of the lumen, with the conduit being reduced in size at a second opening at a second end that is directed distally toward the lumen, and through which first opening to receive in series a point of a removable insertion needle and a probe tip of a measurement device to facilitate guiding either of the point and the tip into the proximal end of the cannula. Desirably, the entry orifice of the guide structure is large, compared to the probe tip of a measurement device, to operate as a large and forgiving target. Furthermore, the guide structure should operate to route the probe tip toward the cannula entrance orifice, even if an axis of the measurement device's probe tip is disposed at an angle compared to the axis of the cannula. Desirably, the guide structure can accommodate misalignment angles of 10, 15, 20, or more degrees, and still assist insertion of the probe tip into the cannula.

Preferred guide structure includes a separate component received by, and affixed in position with respect to, structure associated with the housing. Guide structure desirably provides a guiding surface with improved resistance to needle point sticks compared to material forming the housing. An operable guide can be made, at least in part, from metal. A portion of the guide is usually arranged to provide a funnel-like shape. One operable guide can be characterized as having a fan shape in a cross-section in a plane passing through an axis of the guide. It is desirable for a guide to be arranged to provide a seal surface operable in harmony with structure associated with a measurement device to form an aseptic seal effective to resist passage of contaminant material through the cannula of the introducer.

The introducer is typically installed into a medical patient with an insertion needle assembly adapted to support the cannula for subcutaneous insertion of a distal end portion of the cannula into the patient. The needle assembly is arranged in releasable cooperation with the housing to permit withdrawal of a needle from an installed cannula to dispose a proximal end of the lumen in open communication with the subcutaneous site inside the patient.

Desirably, a one-way coupling is formed, between structure associated with a measurement device and structure associated with the introducer, to resist withdrawal of an installed device's probe tip portion from the introducer's cannula. A one-way coupling permits tool-free connection of two components, but resists tool-free separation of those components. A preferred measurement device carries structure arranged to form a one-way coupling with structure associated with an introducer's housing.

A workable one-way coupling can be formed by capture structure arranged to engage held structure subsequent to forming the desired connection between components. Capture structure can include a hook carried by one of the measurement device and the housing; and a wall associated with a biasing element carried by the other of the device and the housing, with the wall and biasing element being arranged operably to engage the hook to form the one-way coupling. In a preferred embodiment, the measurement device carries a latch element constituting a hook and the housing carries a wall disposed in association with a bridge element. The bridge element biases the wall into capturing engagement with the hook subsequent to assembly of the device into an installed position with respect to the introducer assembly.

Preferably, subsequent to engagement of the one-way coupling, structure is disposed to support the latch and resist a deflection of the latch to resist disengagement of the one-way coupling. In one embodiment, hub structure is arranged to provide such latch support. The hub effectively prevents displacement of the latch sufficient to release the hook portion from the socket structure in which the hook is received.

It is desirable for a coupling between an introducer and a measurement device to be effected simply by sliding the components together. The currently preferred latch element includes a tapered portion arranged to cause a transverse displacement of the bridge element operable to permit the latch element to slide forward axially into place for reception in the socket. The bridge is biased to resist displacement, in a direction caused by the tapered portion, and is operable therefore to capture the latch in engagement in the socket. The measurement device can further provide bridge guard structure arranged to resist tool-free displacement of the bridge element subsequent to engagement of the one-way coupling.

An aseptic seal desirably is formed in an installed biosensor assembly. One workable seal is formed by a seal element being placed into compression between structure of the measurement device and structure associated with the housing. Compression stress in the seal element, operable to form the aseptic seal, can be effected by insertion of a portion of the device into reception in the housing. Compression stress in the seal element beneficially can be maintained by engagement of structure coupling the device and the housing together. The aseptic seal forms a barrier operable to resist passage of contaminants through the lumen subsequent to installing a measurement device.

In a currently preferred embodiment, a barrier includes a seal element placed into compression between structure associated with the housing and structure associated with a biosensor. A probe portion of the biosensor is held at its proximal end by a hub of the biosensor. A seal element, typically carried at a distal end of the hub, is placed into contact with a sealing surface associated with a guide element, carried by the housing, during attachment of the biosensor to the housing. The formed barrier is then effective to block an annulus disposed between the lumen and a circumference of a shaft of the probe portion. Probes are generally sealed along their exterior surface, providing an effective plug to the inner portion of the annulus. In an alternative arrangement, the portion of the probe that is disposed inside the annulus can be blocked by a sealing substance. A coupling arrangement, between a probe and a housing, desirably is provided to maintain a compression load on the assembled seal element. In one suitable arrangement, a hook portion of a latch element of coupling structure is received in contact with a wall of a socket to maintain a compression load on the assembled seal element.

Certain biosensor assemblies within the ambit of certain embodiments of the invention complete an electric path between an electrode and system electronics only subsequent to installation of the biosensor device into an introducer. One such measurement device includes a probe tip portion adapted for insertion through an introducer's cannula for disposition at a subcutaneous site. The cannula carries a first electrode, and the probe tip carries a second electrode. When the device is placed into mating reception in the introducer, a switch element completes an electrically conductive path between the first electrode and an element of a sensor cable carried by the device. The conductive path from the first electrode includes a conductive path disposed along the cannula, which is placed into electrical communication with a wedge element upon assembly of the cannula into a housing. The path continues through a switch arrangement, which can be formed by a conductive strip that is biased into contact with a conductive element that can be arranged as a plate and is carried by the device, at an assembled configuration of the device with respect to holding structure of the housing.

The invention can be embodied as an introducer system, for inserting a probe into a subcutaneous target site in a subject, including an introducer assembly and a biosensor. An introducer includes a cannula having a lumen extending axially between a proximal end and a distal end. The proximal end is associated with affixing structure adapted to resist its motion relative to a skin surface of the subject. The introducer also includes a housing associated with the proximal end of the cannula. The housing provides holding structure configured to interface separately with a needle assembly and a biosensor.

The needle assembly includes an extended needle shaft configured for placement through the lumen to dispose a needle point distally to the distal end of the cannula. The needle shaft is structured to facilitate subcutaneous insertion of the distal end of the cannula into the subject. Also, a portion of the needle assembly is generally structured and arranged in harmony with a portion of the holding structure to effect a releasable coupling therebetween to permit withdrawal of the needle shaft subsequent to placement of the distal end of the cannula in proximity to the subcutaneous site.

A biosensor for adapted for use in the introducer system includes a proximal probe end spaced apart from a distal probe end. The distal probe end is structured for sliding installation through the lumen to the subcutaneous target site. The proximal probe end also desirably is associated with structure adapted to form a one-way coupling with structure associated with the holding structure effective to resist withdrawal of the distal probe end from the cannula.

A method for installing an element of a biosensor into a subcutaneous site in a subject includes the steps of: providing a cannula having a lumen extending between a proximal end and a distal end, with the proximal end being associated with holding structure adapted to interface with an inserter needle assembly and containing an extended needle shaft of that inserter needle assembly; using the inserter needle assembly to insert the distal end of the lumen into proximity to the site; withdrawing the needle shaft from the lumen to provide open communication through the lumen to the site; affixing structure associated with the proximal end of the cannula to a skin surface of the subject; and inserting a probe tip of a measurement device into the lumen and bringing the device into seated engagement with the holding structure to form a one-way connection operable to resist tool-free removal of the probe's tip from the lumen. Sometimes, bringing the biosensor into seated engagement completes an electrically conductive path between an electrode associated with the cannula and an element of a sensor cable associated with the biosensor device. In some cases, bringing the device into seated engagement also, or alternatively, compresses a seal element to form an aseptic seal operable to resist passage of pathogens through the cannula.

### BRIEF DESCRIPTION OF THE SEVERAL VIEWS OF THE DRAWINGS

In the drawings, which illustrate what is currently regarded as the best mode for carrying out the invention:

FIG. 1 is an exploded perspective side view in elevation of a sensor and introducer assembly of a currently preferred embodiment;

FIG. 2a is a top view of the introducer housing and illustrated in FIG. 1;

FIG. 2b is a rear view of the introducer housing illustrated in FIG. 2a;

FIG. 2c is a side view of the introducer housing illustrated in FIG. 2a;

FIG. 2d is a perspective side view in elevation of the introducer housing illustrated in FIG. 2a;

FIG. 2e is a partial cross-sectional side view of the introducer housing illustrated in FIG. 2a, taken through section A-A and looking in the direction of the arrows, and further including a portion of an exemplary probe installed in that housing;

FIG. 3a is a top view of the needle introducer assembly illustrated in FIG. 1;

FIG. 3b is a rear view of the needle introducer assembly illustrated in FIG. 3a;

FIG. 3c is a side view of the needle introducer assembly illustrated in FIG. 3a, now illustrated upside-down;

FIG. 3d is a perspective side view from below of the needle introducer illustrated in FIG. 3a;

FIG. 4a is a top view of the biosensor assembly illustrated in FIG. 1;

FIG. 4b is a rear view of the sensor assembly illustrated in FIG. 4a;

FIG. 4c is a side view of the sensor assembly illustrated in FIG. 4a;

FIG. 4d is a perspective side view in elevation of the sensor assembly illustrated in FIG. 4a;

FIG. 5a is a plan view of an alternative sensor assembly carrying both electrodes on the sensor probe; and

FIG. 5b is a cross-section view, taken through a section illustrated as B-B in FIG. 4c, and looking in the direction of the arrows, but actually taken through the device illustrated in FIG. 5a.

### DETAILED DESCRIPTION OF THE ILLUSTRATED EMBODIMENTS

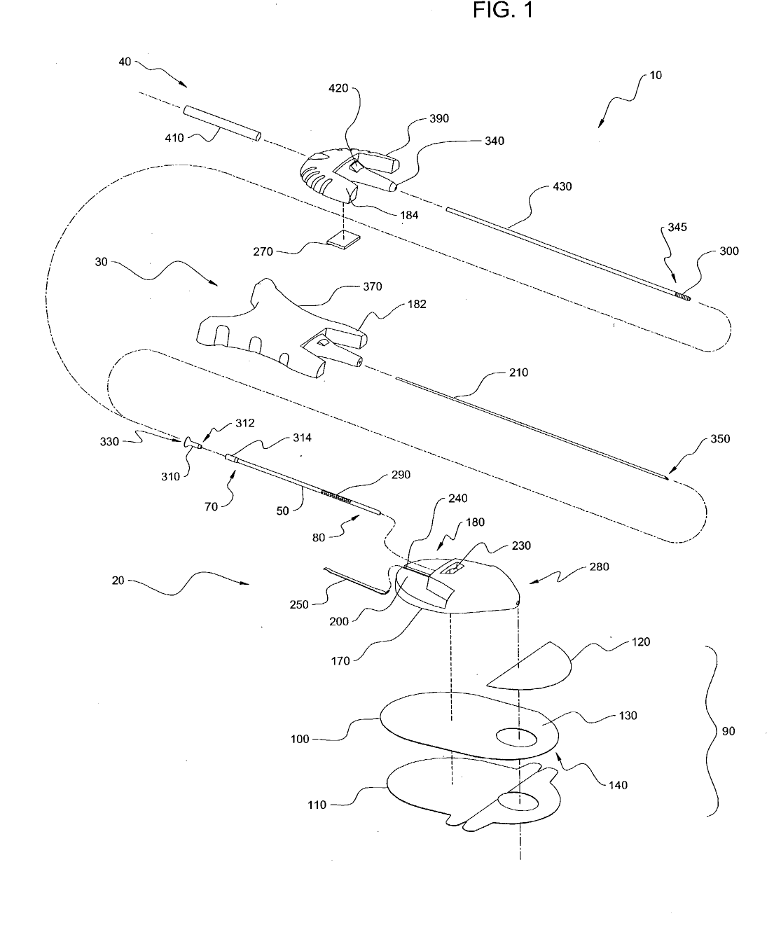

Illustrated in FIG. 1 is a currently preferred embodiment in which an implantable biosensor system, generally 10, comprises an introducer assembly, generally indicated at 20, a needle assembly, generally indicated at 30 and a sensor assembly or measurement device, generally indicated at 40.

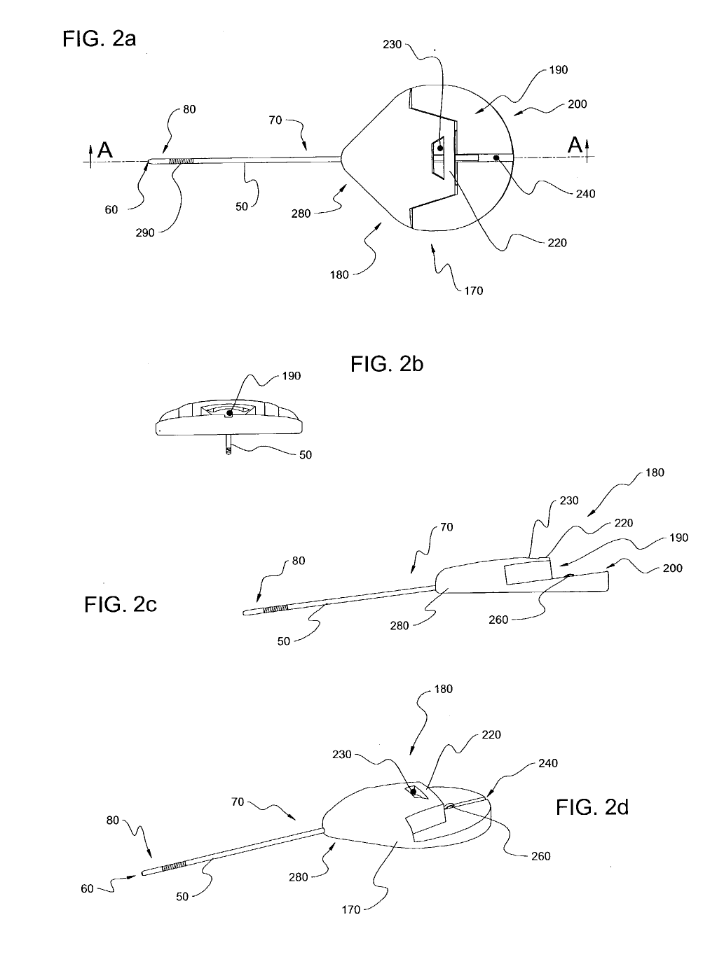

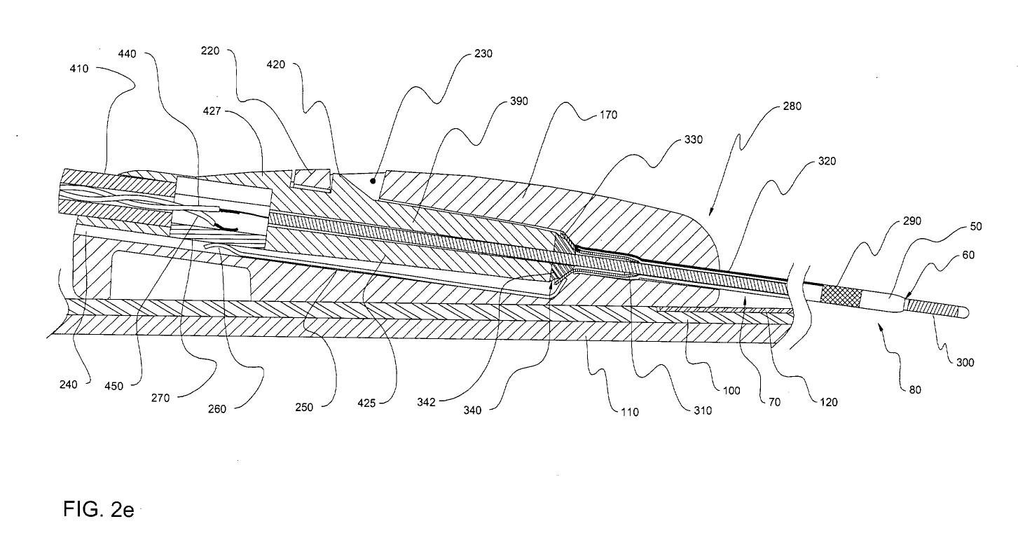

The introducer assembly 20, more fully illustrated in FIGS. 2a-2d, includes an introducer cannula or catheter 50 with a lumen 60 extending between a proximal end 70 and distal end 80 along an axis A-A. Operable cannulas 50 can be manufactured from a variety of materials, including without limitation: Teflon, polyurethane, nylon, and polyvinylchloride (PVC). Some sort of affixing structure, one embodiment of which is indicated generally at 90 in FIG. 1, typically is provided to resist motion of the proximal end 70 relative to a skin surface of a subject (not shown) to maintain a cannula 50 in an installed position.

The illustrated affixing structure 90 includes an adhesive patch 100, release liner 110 and a clear cover 120. Patch 100 can be made from a nonwoven polyester carrying an acrylic skin-compatible adhesive. An operable release liner 110 is formed from siliconized craft paper. The adhesive patch 100 receives the clear cover 120 on an adhesive patch upper surface 130 and receives the release liner 110 on an adhesive patch opposite surface 140. It has been determined that an operable cover 120 may be made from a clear polyurethane. Typically, a cannula 50 carried on a needle 210 is simply poked through the cover 120 during manufacture of an introducer assembly 20. An adhesive, such as a urethane-based adhesive, can be applied to a bottom of housing 170 to affix the top surface 130 of adhesive patch 100 to the housing 170. An alternative coupling approach incorporates an ultrasonic bond between the housing 170 and the patch 100.

Prior to being applied to the skin of a subject, the release liner 110 is removed from the sticky adhesive patch opposite surface 140 preparatory to adhering the opposite surface 140 to the skin of the subject. Other arrangements for affixing structure are within contemplation, including application of adhesive tape to maintain a proximal end 70 in a position with respect to a skin surface. Such adhesive tape may adhere to cannula 50, and/or to a cannula holding structure affixed at proximal end 70, and to the skin of a subject. As an other alternative, adhesive material may be applied to a bottom surface of a cannula holding structure to permit direct attachment of that holding structure to a skin surface.

An introducer housing, indicated generally at 170 in FIG. 2a, is affixed to a proximal end of cannula 50, and is one example of a cannula holding structure. Housing 170 desirably is a relatively low-profile structure, having a base that is wide compared to the thickness of the housing 170. A generalization can be made that the housing's average thickness usually is less than about one-fourth the maximum width of the bottom surface. The low-profile, substantially planar shape provides a large area over which to distribute loads and thereby reduce patient discomfort resulting from wearing the device. A low profile desirably can reduce snagging and bumping of the attached introducer assembly 20. The large bottom surface area of a housing 170 also facilitates its adherence to a patch, or affixing structure 90.

Housing 170 also provides holding structure, generally indicated at 180, that is operable to interface with structure carried by an insertion or installation needle or by a biosensor or other measurement device. Holding structure 180 desirably includes an enlarged opening 190 (FIGS. 2b,c) at a proximal holding end 200. The illustrated opening 190 is in communication with, and in general, narrows distally toward the lumen 60. The illustrated opening 190 leads to conduit structure configured somewhat like a funnel, and can operate as a guide structure to facilitate insertion of distal ends of either a removable needle 210 or a selected probe, such as sensor extension 430 illustrated in FIG. 1, into the proximal end 70 of a cannula 50. An exemplary needle 210 can be made from stainless steel, or a material with sufficient rigidity and point-holding capacity.

Desirably, the entry orifice 190 is large, compared to the probe tip of a measurement device, to operate as a large and forgiving target to facilitate inserting a biosensor into the cannula 50 by a user who is visually impaired, or is infirm, or otherwise lacking in hand-eye coordination or dexterity. Furthermore, the funnel-like guide structure should operate to route the probe tip toward the cannula entrance orifice, even if an axis of the measurement device's probe tip is disposed at an angle compared to the axis of the cannula. Desirably, the guide structure can accommodate such misalignment entrance angles of 10, 15, 20, 30, and even up to 45 or more degrees in the X-Y plane of the housing 170's width, and still assist insertion of the probe tip into the cannula. A similar degree of freedom typically is provided in a direction corresponding to a Z axis, but, of course, above the skin surface on which the introducer 20 is installed. Therefore, a 3-dimensional half-conic envelope is provided by the introducer in which envelope a probe can successfully be inserted into engagement within the opening 190. Flexible probes tend to follow a path established by conduit structure of the funnel-like guide toward the entrance of the cannula 50. In a preferred embodiment, extended hub structure 425 (FIG. 2e) acts as a secondary guide to further align the sensor 40 and the housing 170 to help accomplish coplanar axial coupling between structure associated with a housing 170 and a measurement device 40. The preferred axial coupling also avoids twisting between the components, such as might be required to secure a luer-locking type of joint.

In general, a housing 170 associated with an introducer's cannula 50 can be one component or an assembly of a plurality of components. Typically, a housing 170 includes a frame component that is injection molded from polypropylene, polyethylene, or polycarbonate, although other medical-grade plastics can also be used. Such a housing 170 typically provides structure operable to anchor the proximal end of the cannula 50 with respect to a skin surface of a subject. Desirably, the housing 170 also provides a guide structure operable to facilitate insertion of a needle or probe tip into the proximal end of the cannula 50. Preferred guide structures provide an enlarged opening 190 operable as a target that is larger in size than an opening at the proximal end of the cannula 50.

With particular reference to FIGS. 2a through 2d, holding structure 180 is generally operable to provide a connection interface between an introducer assembly 20 and either of a needle assembly 30 or a measurement device 40. Desirably, a housing 170 and an inserted needle assembly 30, or an inserted measurement device 40, forms a low-profile, substantially coplanar, axially engaged, connection. Illustrated holding structure 180 is arranged in harmony with paired outrigger arms 182 and 184 to resist rotation between a needle assembly 30, or a measurement device 40, and the introducer assembly 20. Holding structure 180 typically is arranged to provide a stable interface for rotation control during insertion and removal of a needle 210, and removal of a cannula 50 from a subject.

Certain preferred holding structure 180 includes structure operable to form a one-way coupling with structure carried by a measurement device 40. One preferred arrangement of such coupling structure includes a bridge 220 and an adjacent opening or socket 230 for selected interaction with cooperating structure carried by a sensor 40. The opening 230 can provide visual feedback to show proper, fully seated, assembly of an installation needle or of a biosensor in the introducer assembly 120.

The illustrated housing 170 further includes a channel 240 structured and arranged to accommodate an electrically conductive strip 250 (FIGS. 1 and 2e). A raised conductive portion 260 of the illustrated conductive strip 250 is positioned in the channel 240 to abut a conductive plate 270 associated with any of various selected probes to be stationed in the holding structure 180 and seated within the funnel opening 190. The conductive strip 250 and plate 270 maybe formed from a metal material, although any electrically conductive material can be employed. In a preferred embodiment, conductive strip 250 and plate 270 are made from a Beryllium-Copper alloy.

The proximal end 70 of the cannula or catheter 50 is attached to a distal housing end 280 of the housing 170. The cannula or catheter 50 in a currently preferred embodiment, perhaps best shown in FIGS. 2a and 2e, includes a reference electrode 290 arranged to cooperate with a working electrode 300 such as those used with implantable biosensors for continuous glucose monitoring in the context of diabetes therapy. However, it is recognized that such electrodes 290, 300 may both be located on a sensor probe installed through a catheter 50 rather than including the reference electrode on the delivery catheter 50. Furthermore, the location of such electrodes can be reversed, if desired. That is, a reference electrode can be located on a probe tip, and a working electrode can be located on a cannula 50.

The holding structure 180, as illustrated in FIG. 2e, includes an electrically conductive catheter wedge 310 anchored within the funnel opening 190 of the holding structure 180. In one method of assembling a introducer assembly 20, a distal end of wedge 310 is first forced into engagement inside of enlarged neck portion 312 of a catheter 50. Then, the thus-formed assembly is inserted through opening 190 into the housing 170. A portion of housing 170 desirably is adapted to receive neck 314 in close radial engagement, and to provide a distal restriction that is smaller in diameter than neck 314. The catheter assembly can then be held in place inside housing 170 with any operable retaining structure, including without limitation: a press-fit, adhesive joint, or frictionally induced bond. In a preferred embodiment, a friction-fit is provided between housing 170 and neck 314. It is currently preferred for the wedge 310 to be sufficiently rigid to resist separation of the cannula 50 from engagement in a housing 170 as the introducer assembly 20 is withdrawn from a subject.

An exemplary wedge 310 also provides a guiding, funnel-like opening operating in harmony with opening 190 to receive a probe tip 345, or needle tip 350, to facilitate entrance of such tip 345, 350 into lumen 60 inside cannula 50. As illustrated, the catheter wedge 310 is situated to bring the conductive strip 250 and an electrical lead 320 of the reference electrode 290 into electrical communication; one with the other. Electrical lead 320 extends from the electrode 290 to the proximal end 70 of the catheter 50, where it wraps sufficiently to contact the wedge 310. It currently is preferred to make electrical leads 320 from silver plated stainless steel. However, copper, bare stainless steel, solid silver, or other electrically conductive materials are also operable.

The illustrated catheter wedge 310 provides a third function in that it also provides a proximal-facing sealing surface 330 against which a distal face 340 of any of various selected probes may establish an aseptic seal to resist passage of pathogenic organisms. Seal 342 typically is a resilient element capable of conforming to surface 330 under a compressive load to form a barrier to passage of contaminants through the cannula 60. One exemplary material that is operable as a seal element is silicone rubber.

A guide structure 310 can be a separate component, or it can be substituted by, or inherent in, funnel 190. In the preferred embodiment, guide 310 is formed from a metallic or other electrically conductive material. It is desirable for guide 310 to provide a relatively large entrance opening to form an easy target for needle or probe distal tips. Operable guides 310 can be substantially symmetrical in revolution about a central axis (such as the funnel shape illustrated in FIG. 1). Operable guides 310 maybe embodied more 2-dimensionally, such as in the shape of a fan. In any case, guide 310 and funnel opening 190 are arranged to provide a target that is larger than a probe tip 345. The proximal opening defined by such components can be 2, 5, 10, 20, or more, times as large as a diameter of a probe tip 345. It should be noted that housing 170 typically is injection molded. Guide 310 and funnel 190 are arranged to provide a target larger than an opening inherent in a draft requirement to remove a catheter core element from the injection mold.

It is desirable for guide 310 to provide a surface that is resistant to needle sticks, and arranged to urge a tip 350 of a needle 210 to slide towards a proximal entrance of a cannula 50. Needle stick resistance can be developed by arranging the proximal surface 330 at an oblique angle to an approaching needle point and/or by surface hardness.

It is currently preferred for guide 310 to be arranged as a 3-D funnel, because proximal surface 330 also acts as seal surface. While not required, an interface that is independent upon orientation simplifies manufacturing complexity and forming an aseptic seal to resist passage of contaminants through the cannula 50. Contaminants can be regarded as externally generated or internally generated. External contaminants can include germs, fluids, and debris. Internal contaminants may include body fluids.

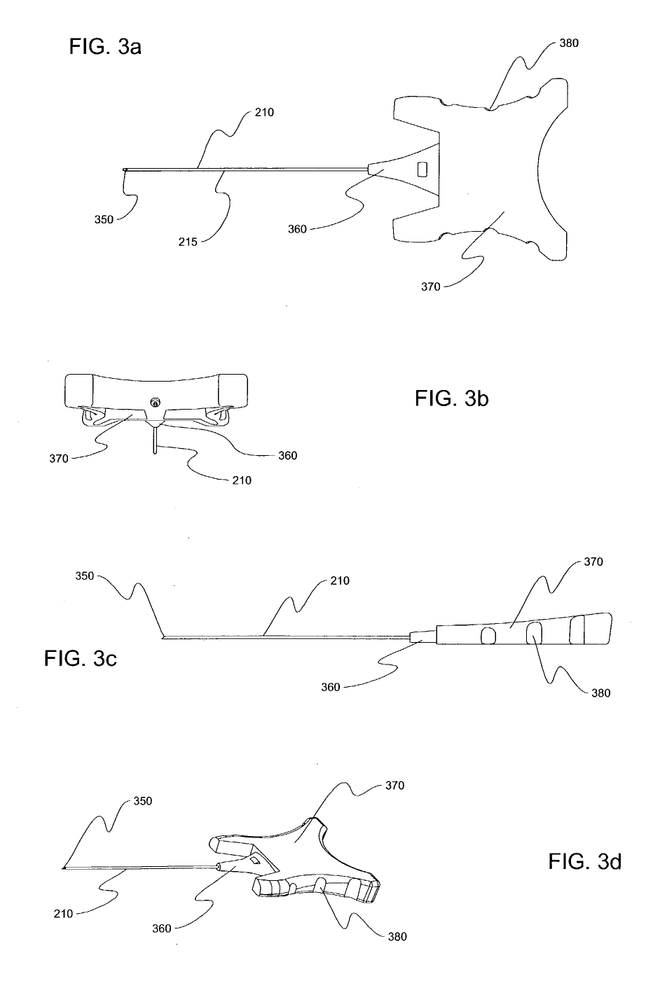

Prior to introduction of a given selected probe into the cannula or catheter 50 at a patient treatment site, the needle assembly 30 is positioned to install the cannula 50 into the patient's subcutaneous tissue. An exemplary needle assembly 30, depicted in detail in FIGS. 3a-3d, is received through the funnel opening 190 of the holding structure 180 for entrance into the catheter lumen 60. When the needle assembly 30 is placed into a catheter installation position with respect to holding structure 180, needle point 350 extends distally beyond and outside of the distal end 80 of the catheter 50. Also, illustrated releasable seating structure 360 is located within the funnel opening 190 of the holding structure 180.

A cannula 50 is regarded as being installed onto a needle assembly 30 when the cannula 50 is located in a position where the needle 210 of a needle assembly 30 provides sufficient support to the cannula 50 to permit sticking the distal portion of the cannula 50 into the subcutaneous area of a subject. A cannula 50 is regarded as being installed in a subject when the distal portion of the cannula 50 is placed into the subcutaneous area, and the proximal end of the cannula 50 is affixed to resist its motion relative to the skin surface of the subject.

A needle connector 370, which may have ridges 380 formed on its side for better gripping, facilitates handling of the needle assembly 30 in conjunction with the introducer assembly 20. The introducer assembly 20, with its relatively less rigid cannula 50 containing and substantially supported by the relative more rigid needle 210 and with the extending needle point 350, is rendered more readily deliverable to a subcutaneous patient site. Upon delivery of the introducer assembly 20 to the patient site and anchoring of the introducer assembly 20 appropriately, such as with the affixing structure 90, the needle assembly 30 is grasped by the needle connector 370 along the ridges 380 and retracted from the introducer assembly 20. Using sterile technique, a user may then readily in sequence introduce a selected probe into the catheter 50 and into the patient.

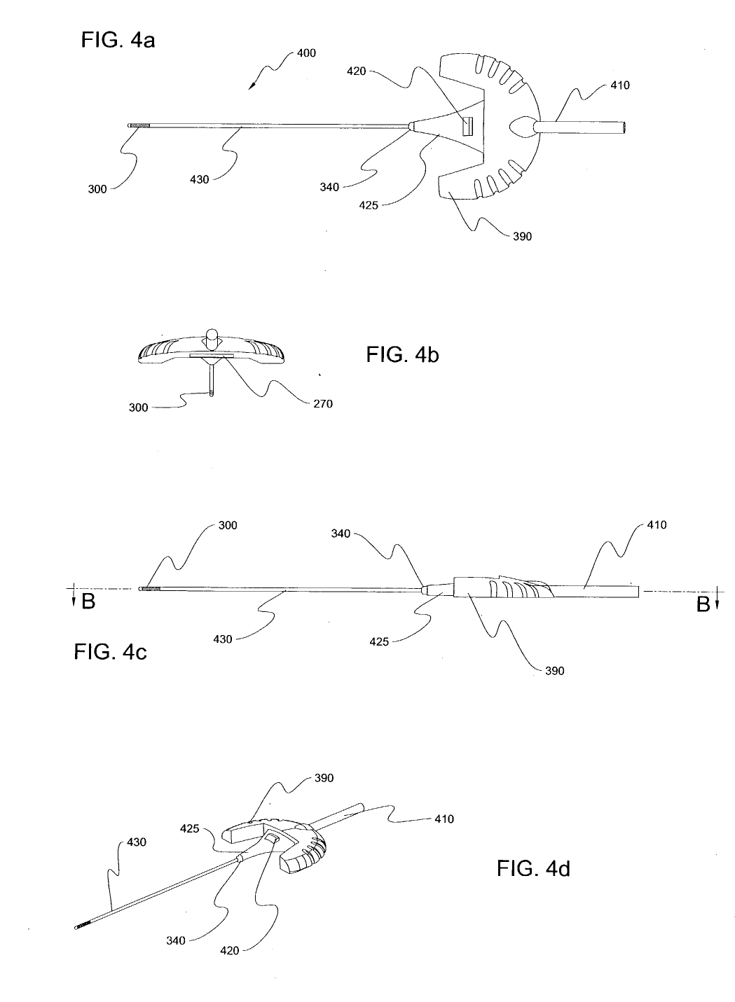

Illustrative of such a selected probe is the sensor assembly 40 of FIGS. 4a-4d. The sensor assembly 40 includes a sensor connector 390, a sensor probe, generally 400, and a sensor cable 410. While only a terminal portion of cable 410 is illustrated, sensor cable 410 typically connects the sensor assembly 20 with system electronics (not shown). In the currently preferred embodiment illustrated in FIG. 2e, an electrical connection is formed between one conductive lead of the sensor cable 410 and the conductive plate 270.

The sensor connector 390, unlike the releasable seating structure 360 of the needle connector 370, desirably is equipped with structure operable to form a one-way connection to structure associated with the housing 170. An exemplary capture arrangement is illustrated as tapered latch 420 structured and arranged to deflect the bridge 220 of the introducer housing 170 upon distal passage of hub structure 425 of the sensor connector 390 into the funnel opening 190 of the holding structure 180. The thus captured latch 420, while engaged within the opening or socket 230 of the holding structure 180, prevents retraction of the sensor connector 390 and its associated sensor 430 from the holding structure 180. A captured latch 420 acts as a hook engaging a wall of socket 230. The captured latch 420 also maintains a compressive load on seal element 342 to maintain the aseptic seal. It is also within contemplation that hub 425 can be structured directly to be operable as a seal element.

As illustrated in FIG. 2e, a safety barrier or bridge guard structure 427 can be arranged to block proximal access to bridge 220 operable to resist tool-free deflection of the bridge to resist removal of an installed device 40. Furthermore, hub 425, in harmony with structure associated with housing 170, provides a support to resist deflection of an installed latch 420 operable to resist disengagement of the one-way coupling.

Of course, opening 230 would be equally effective if arranged as a covered socket in which to receive latch 420. Formation of capture element 230 as an opening passing through bridge 220 affords some manufacturing advantage by reducing mold complexity. In any event, retraction of a captured sensor assembly 40 from a treatment site necessarily results in concurrent retraction of the entire introducer assembly 20 from the site.

Bridge 220 acts as a biasing element operable to capture latch 420 in socket 230 once device 40 is fully seated in housing 170. Bridge 220 resists displacement in a direction caused by a distal tapered portion of latch 420. Of course it is recognized that arrangements of bridge and latch type of structures, other than that illustrated, are operable to form the desired one-way coupling between an introducer assembly 20 and a device 40. Operable arrangements simply cause a structural interference between the respective components.

Still with reference to FIG. 2e, a sensor cable 410 typically contains at least a pair of wires for transmittal of electric signals from a sensor. As illustrated, a working electrode wire 440 and a reference electrode wire 450 connect the working electrode 300 and the reference electrode 290 respectively with the system electronics (not shown). System electronics typically can include elements for signal amplification, sensor polarization, data display, data storage and data download.

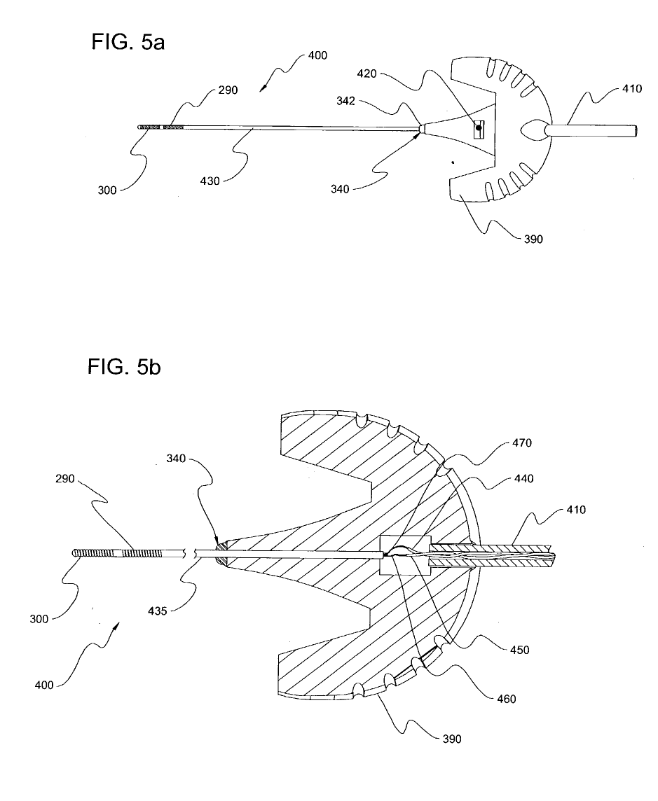

Still with reference to FIG. 2e, conductive plate 270 is one arrangement of structure operable as a portion of an electrical path between an electrode carried by cannula 50 and a conductive element of a sensor cable 410. Conductor 250 and plate 270 operate as a switch that is closed when a measurement device 40 is installed in an introducer assembly 20. Other conductive path arrangements are within contemplation, including routing a reference electrode lead 460 or reference electrode lead 470 (see FIG. 5b) to terminate at a distal portion of a sensor connector 390 for direct contact to a guide funnel or wedge 310.

As illustrated in FIG. 5b, an alternative sensor extension 435 carries a pair of electrodes; both a working electrode 300 and a reference electrode 290. Of course, the position of working electrode 300 and reference electrode 290 can be reversed from the illustrated arrangement. In a sensor assembly 40 having a probe extension arranged similar to extension 435, no switch is closed upon insertion of the sensor 435 into an introducer housing 170. Instead, the electrode wires, 440 and 450 respectively, already are in electrical communication with the electrode leads, 460 and 470, respectively.

Electrode wires 440, 450, typically are soldered to the electrode leads 460, 470, and the resulting connection may then be coated or encapsulated with a nonconductive material. A similar approach typically is employed to join reference electrode wire 450 to conductive plate 270. In any case, care is taken to avoid forming a short-circuit between the two types of wires and/or leads. One preferred affixing arrangement includes applying an epoxy coating over the solderjoint. Other affixing arrangements to join leads to wires are within contemplation, including use of a crimp-type joint, or an electrically conductive adhesive, rather than soldering.

In a method of installing the sensor system of the present invention, an inserter needle assembly 20 is used to insert a distal end 80 of a catheter lumen 60 into proximity to a subcutaneous site in a subject (or medical patient), where the property or physiological state is to be measured. Then, the needle shaft 215 is withdrawn from lumen 60 to provide open communication through lumen 60 for access by a probe element (such as an electrode 300) of a measurement device 10 to the site. Structure associated with the proximal end of the cannula is affixed to a skin surface of the subject to hold the cannula in the installed position. A probe tip 345 of a measurement device can then be inserted into the lumen 60 and the measurement device 40 brought into seated engagement with holding structure 180 to form a one-way connection operable to resist tool-free removal of the probe tip 345 from lumen 60. In certain embodiments of a probe system 10, bringing the sensor assembly 40 into such seated engagement in the introducer assembly 20 forms an aseptic seal to resist passage of contaminants, or harmful substances, through the cannula 60. In other preferred systems 10, forming the seated engagement between a sensor assembly 40 and the introducer assembly 20 can also operate as closing a switch to complete an electrically conductive path between an electrode associated with the cannula 60 and a sensor wire, 440 or 450, of a sensor cable 410 associated with the sensor assembly 40.

The system, apparatus and method of the present invention provide distinct advantages over prior sensor introducer systems. Thus, reference herein to specific details of the illustrated or other preferred embodiments is by way of example and not intended to limit the scope of the appended claims. It will be apparent to those skilled in the art that modifications of the basic illustrated embodiments may be made without departing from the essential spirit and scope of the invention as recited by the following claims.

What is claimed is:

1. An introducer, to provide subcutaneous access into a subject for a measurement device, said introducer comprising:
a cannula having a lumen extending between proximal and a distal ends, said proximal end being associated with affixing structure adapted to resist motion of said proximal end relative to a skin surface of the subject; and
a substantially planar housing having a bottom surface and a thickness, disposed substantially transverse to said bottom surface, that is less than about one-fourth the size of a maximum width of said bottom surface, said housing being arranged to dispose a first guide structure in association with said proximal end of said lumen, said first guide structure providing a conduit having a first opening arranged to provide a forgiving target, said first opening being disposed at a first end of said conduit and being sized larger than a size of an opening at said proximal end of said lumen, said conduit being reduced in size at a second opening at a second end that is directed distally toward said lumen, and through which first opening to receive in series a removable insertion needle point and a probe tip of a measurement device to facilitate guiding said tip into said proximal end of said cannula.

2. The introducer of claim 1, in combination with a measurement device, wherein:
said measurement device comprises a second guide structure arranged in harmony with said first guide structure and adapted to align an inserted measurement device with said housing for axially directed, snap-fit coupling of said measurement device to structure associated with said housing.

3. The introducer of claim 1, wherein:
said first guide structure comprises a guide surface having improved resistance to needle point sticks compared to material forming said housing.

4. The introducer of claim 1, wherein:
said first guide structure accommodates a misaligned approach angle of said measurement device in excess of 15 degrees.

5. The introducer of claim 1, wherein:
said first guide structure accommodates a misaligned approach angle of said measurement device of up to about 45 degrees.

6. The introducer of claim 1, wherein:
a portion of said first guide structure is arranged to provide a fan shape in a cross-section in a plane passing through an axis of said guide structure.

7. The introducer of claim 1, wherein:
a portion of said first guide structure is arranged to provide a seal surface operable in harmony with structure associated with said device to form an aseptic seal effective to resist passage of contaminant material through said cannula.

8. The introducer of claim 1, in combination with:
an insertion needle assembly adapted to support said cannula for subcutaneous insertion of a distal end portion of said cannula into the subject, and arranged in cooperation with said housing to permit withdrawal of a needle from an installed said cannula to dispose said lumen in open communication with a subcutaneous site in the subject; and
a measurement device comprising a probe tip portion adapted for insertion through said cannula for disposition at said site; wherein said measurement device carries structure arranged to form a one-way coupling effective to resist withdrawal of said probe tip portion from said cannula.

9. The combination of claim 8, wherein:
said one-way coupling is formed between structure associated with said device and structure associated with said housing.

10. The introducer of claim 1, in combination with:
an insertion needle assembly adapted to support said cannula for subcutaneous insertion of a distal end portion of said cannula into the subject, and arranged in cooperation with said housing to permit withdrawal of a needle from an installed said cannula to dispose said lumen in open communication with a subcutaneous site in the subject; and
a measurement device comprising a probe tip portion adapted for insertion through said cannula for disposition at said site; wherein structure associated with said housing is arranged to cooperate with structure associated with said device to form an aseptic seal operable to resist passage of contaminants through said lumen.

11. The combination of claim 10, wherein:
said seal is formed by a seal element being placed into compression between structure of said device and structure associated with said housing.

12. The combination of claim 11, wherein:
compression stress in said seal element, operable to form said seal, is effected by insertion of a portion of said device into reception in said housing.

13. The combination of claim 11, wherein:
compression stress in said seal element is maintained by engagement of a one-way coupling between said device and said housing.

14. The introducer of claim 1, in combination with:
a measurement device comprising a probe tip portion adapted for insertion through said cannula for disposition at said site; wherein:
said cannula carries a first electrode;
said probe tip comprises a second electrode; and
assembly of said device into mating reception in holding structure carried by said housing completes an electrically conductive path between said first electrode and an element of a sensor cable carried by said device.

15. The combination of claim 10, wherein:
said measurement device carries structure arranged to form a one-way coupling effective to resist withdrawal of said probe tip portion from said cannula.

16. The combination of claim 10, wherein:
said cannula carries a first electrode;
said probe tip comprises a second electrode; and
assembly of said device into mating reception in holding structure carried by said housing completes an electrically conductive path between said first electrode and an element of a sensor cable carried by said device.

17. The combination of claim 15, wherein:
said cannula carries a first electrode;
said probe tip comprises a second electrode; and
assembly of said device into mating reception in holding structure carried by said housing completes an electrically conductive path between said first electrode and an element of a sensor cable carried by said device.

18. The combination of claim 8, wherein:
said cannula carries a first electrode;
said probe tip comprises a second electrode; and
assembly of said device into mating reception in holding structure carried by said housing completes an electrically conductive path between said first electrode and an element of a sensor cable carried by said device.

19. An introducer system for a biosensor, said introducer system comprising:
an introducer assembly comprising a cannula having a lumen extending between proximal and distal ends, said proximal end being associated with a housing that carries affixing structure adapted to resist motion of said proximal end relative to a skin surface of a subject, said housing further carrying holding structure configured to interface individually in series with first structure associated with an installation needle and with second structure associated with said biosensor;
an installation needle assembly comprising said first structure and said installation needle, said needle being structured for insertion into said lumen to facilitate installation of a distal end of said cannula into a subcutaneous target site of the subject, said needle being removable from said cannula subsequent to placement of said distal end of said cannula at said subcutaneous target site; and
a biosensor comprising said second structure and a probe portion configured and arranged to permit a distal probe end to slide through said lumen to dispose said probe end at said site when forming an interface between portions of said holding structure and said second structure, said biosensor further being arranged in harmony with said housing to form a barrier operable to resist passage of outer contaminants into said lumen subsequent to forming said connection.

20. The introducer system of claim 19, wherein:
said barrier comprises a seal element placed into compression between structure associated with said housing and structure associated with said biosensor.

21. The introducer system of claim 19, wherein:
said probe portion is held at a proximal end by a hub of said biosensor; and
said barrier comprises structure, carried at a distal end of said hub, that is placed into contact with a sealing surface associated with said housing during attachment of said biosensor to said housing, said barrier being effective to block an annulus disposed between said lumen and a circumference of a shaft of said probe portion.

22. The introducer system of claim 19, wherein:
said probe portion comprises a proximal probe end spaced apart from said distal probe end, said proximal probe end being associated with a hub, a distal end of said hub being arranged to form a sealing surface disposed around a circumference of said proximal probe end; and
said holding structure comprises a seal face structured to cooperate with said sealing surface to maintain a seal, operable to resist passage of contaminants through said lumen, subsequent to connection of said second structure to said holding structure.

23. The introducer system of claim 19, further comprising:
capture structure arranged to form a one-way coupling effective to resist withdrawal of said distal probe end from said cannula subsequent to forming said connection between said holding structure and said second structure.

24. The introducer system of claim 23, wherein said capture structure comprises:
a hook carried by one of said biosensor and said housing; and
a wall associated with a biasing element carried by the other of said biosensor and said housing, said wall and biasing element being arranged operably to engage said hook to form said one-way coupling.

25. The introducer system of claim 19, wherein:
said cannula carries a first electrode component of said biosensor;
said distal probe end comprises a second electrode component of said biosensor; and
assembly of said second structure into mating reception in holding structure carried by said housing completes an electrically conductive path between said first electrode and an element of a sensor cable carried by said biosensor.

26. An introducer system for inserting a probe into a subcutaneous target site in a subject,
said introducer system comprising:
an introducer assembly comprising:
a cannula having a lumen extending axially between a proximal end and a distal end, said proximal end being associated with affixing structure adapted to resist motion of said proximal end relative to a skin surface of the subject; and
a housing associated with said proximal end of said cannula and providing holding structure, said introducer assembly being configured to interface separately with:
a needle assembly comprising an extended needle shaft configured for placement through said lumen to dispose a needle point distally to said distal end of said cannula, said needle shaft being structured to facilitate subcutaneous insertion of said distal end of said cannula into the subject, a portion of said needle assembly being structured and arranged in harmony with a portion of said holding structure to effect a releasable coupling therebetween to permit withdrawal of said needle shaft subsequent to placement of said distal end of said cannula in proximity to said site; and
a device comprising a proximal probe end spaced apart from a distal probe end, said distal probe end being structured for sliding installation through said lumen to said subcutaneous target site, said proximal probe end being associated with structure adapted to form a one-way coupling with structure associated with said holding structure effective to resist withdrawal of said distal probe end from said cannula.

27. The introducer system of claim 26, wherein:
said one-way coupling is formed between structure carried by said device and structure carried by said housing.

28. The introducer system of claim 27, wherein:
said device carries a latch element;
said housing carries a wall disposed in association with a bridge element; and
subsequent to assembly of said device into an installed position with respect to said introducer assembly, said latch element is received in contact with said wall.

29. The introducer system of claim 28, wherein:
subsequent to engagement of structure forming said one-way coupling, structure is disposed to support said latch, to resist a deflection of said latch, operable to resist disengagement of said one-way coupling.

30. The introducer system of claim 28, wherein:
said latch element includes a tapered portion; and
during assembly of said device into an installed position with respect to said introducer assembly, said tapered portion causes a displacement of said bridge element operable to permit said latch element to be disposed for reception in said socket.

31. The introducer system of claim 30, wherein:
said bridge is biased to resist displacement, in a direction caused by said tapered portion, operable to capture said latch in engagement in said socket.

32. The introducer system of claim 31, wherein:
said device carries bridge guard structure arranged to resist tool-free displacement of said bridge element subsequent to engagement of said one-way coupling.

33. The introducer system of claim 26, wherein:
said cannula carries a first electrode component of a biosensor;
said distal probe end comprises a second electrode component of said biosensor; and
assembly of said device into mating reception in said holding structure completes an electrically conductive path between said first electrode and an element of a sensor cable carried by said device.

34. An introducer system, for inserting a probe into a subcutaneous target site in a subject, comprising:
an introducer assembly comprising:
a cannula having a lumen extending axially between a proximal end and a distal end, said cannula carrying a first electrode component of a biosensor, said proximal end being associated with affixing structure adapted to resist motion of said proximal end relative to a skin surface of the subject; and
a housing associated with said proximal end of said cannula and providing holding structure, said introducer assembly being configured to interface separately with:
a needle assembly comprising an extended needle shaft configured for placement through said lumen to dispose a needle point distally to said distal end of said cannula, said needle shaft being structured to facilitate subcutaneous insertion of said distal end of said cannula into the subject, a portion of said needle assembly being structured and arranged in harmony with a portion of said holding structure to effect a releasable coupling therebetween to permit withdrawal of said needle shaft subsequent to placement of said distal end of said cannula in proximity to said site; and
a device comprising a proximal probe end spaced apart from a distal probe end, said distal probe end being structured for sliding installation through said lumen to dispose a second electrode component of said biosensor at said subcutaneous target site; wherein:
assembly of said device into mating reception in said holding structure completes an electrically conductive path between said first electrode and an element of a sensor cable carried by said device.

35. The introducer system of claim 34, wherein said conductive path comprises:
a guide element operable to assist insertion of said probe end into said cannula; and
a switch element biased into contact with a conductor, carried by said device, at an assembled configuration of said device and said holding structure.

36. A method for installing an element of a biosensor into a subcutaneous site in a subject, said method comprising:
providing a cannula having a lumen extending between a proximal end and a distal end, said proximal end being associated with holding structure adapted to interface with an inserter needle assembly, said lumen containing an extended needle shaft of said inserter needle assembly;
using said inserter needle assembly to insert said distal end of said lumen into proximity to the subcutaneous site;
withdrawing said needle shaft from said lumen to provide open communication through said lumen to the subcutaneous site;
affixing structure associated with said proximal end of said cannula to a skin surface of the subject; and
inserting a probe tip of a measurement device into said lumen and bringing said device into seated engagement with said holding structure to form a one-way connection operable to resist tool-free removal of said probe tip from said lumen.

37. The method according to claim 36, wherein:
bringing said device into said seated engagement completes an electrically conductive path between an electrode associated with said cannula and an element of a sensor cable associated with said device.

38. The method according to claim 36, wherein:
bringing said device into said seated engagement also compresses a seal element to form an aseptic seal operable to resist passage of pathogens through said cannula.
